(19) United States
(12) Patent Application Publication
(10) Pub. No.: US 2020/0230384 A1
(43) Pub. Date: Jul. 23, 2020

(54) METHOD FOR INTRANASAL STEM CELL THERAPY

(71) Applicant: Joy Kong, Chatsworth, CA (US)

(72) Inventor: Joy Kong, Chatsworth, CA (US)

(21) Appl. No.: 16/253,681

(22) Filed: Jan. 22, 2019

(51) Int. Cl. A61M 31/00; A61M 5/14; A61F 13/36; A61M 19/00; A61M 29/00; A61K 33/14; A61K 31/047; A61K 38/06; A61K 31/167; A61K 31/137; A61K 35/28

(52) U.S. Cl. CPC A61M 31/00; A61M 2205/587; A61F 13/36; A61M 19/00; A61M 29/00; A61K 33/14; A61K 31/047; A61K 38/063; A61K 31/167; A61K 31/137; A61K 35/28; A61M 2210/0618; A61M 2202/09; A61M 2230/205; A61M 2230/30; A61M 2230/50; A61M 5/14

(57) ABSTRACT

A method for intranasal stem cell therapy provides a non-invasive procedure to inject mesenchymal stem cells directly through the nostrils for stem cell deposition in nerve endings, for the uptake of large number of stem cells directly into the central nervous system, and bypassing the blood-brain barrier. The method involves steps: preparing the patient psychologically and physically prior to receiving the stem cell specimen; intravenously introducing a solution of sodium chloride or Ringer's lactate containing mannitol, and glutathione. The method also includes: clearing and illuminating the inferior turbinate of the nostrils; urging a pair of absorbent fiber soaked with lidocaine and epinephrine into the nostrils; inserting, through one of the nostrils, a 27G 1¼″ needle at a midpoint of the inferior turbinate at a 30° angle relative to the facial plane, and at an angle between 5° to 10° relative to a lateral reference point; and performing post-procedure maintenance.

### FIELD OF THE INVENTION

The present invention relates generally to a method for intranasal (IN) stem cell therapy. More so, the present invention relates to a method that provides a non-invasive procedure to inject mesenchymal stem cells directly through the nostrils for stem cell deposition in well-vascularized areas rich in cranial nerve endings, which allows for the uptake of large number of stem cells directly into the central nervous system, and bypasses the blood-brain barrier; whereby the method involves a series of steps to prepare the patient, both mentally and physically, to receive stem cell therapy; conduct intravenous infusion of a substance that helps open up the blood brain barrier; clear the nostrils for introduction of the stem cell specimen; introduce the stem cell specimen at a specific depth and angle through the inferior turbinate of the nostrils; and maintenance/monitor the patient after administration of the stem cell specimen.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Typically, stem cells are primitive cells in our body serving as a reservoir, able to replenish itself and differentiate into a wide range of specialized cells, in order to replace damaged cells and regenerate tissue. Stem cells have innate intelligence, able to home in to injured areas, secrete bioactive molecules that exert local and systemic effects, reduce inflammation, and recruit local cells to assist in the healing process. Mesenchymal stem cells is more potent in many aspects to stem cells in an adult's body. Umbilical cord tissue derived stem cells are particularly rich in mesenchymal stem cells.

The blood-brain barrier (BBB) restricts the use of numerous therapeutic agents that have been developed to treat memory loss and neurodegeneration because it limits CNS penetration, depending on drug size or charge. Although invasive methods of administration (for instance intracerebral or intraventricular) have been used to overcome the BBB, these methods are not practical for use in humans for several reasons, including convenience, safety, and cost.

However, direct delivery of therapeutics from the nasal cavity into the CNS (IN delivery) bypasses the BBB and provides an alternative to invasive methods of drug administration. Noninvasive IN delivery targets therapeutics to the CNS, reducing systemic exposure and side effects; this can be advantageous for delivery of many CNS therapeutics, including those that cannot cross BBB upon systemic administration.

Other proposals have involved methods for administering stem cell specimens. The problem with these methods is that they do not bypass the blood-brain barrier. Also, the prior art methods do not inject stem cells directly through the nostrils for cell deposition in nerve endings. Even though the above cited methods for administering stem cell specimens meet some of the needs of the market, a method for intranasal stem cell therapy that provides a non-invasive procedure to inject stem cells directly through the nostrils for stem cell deposition in nerve endings, and bypasses the blood-brain barrier; whereby the method involves a series of steps to prepare the patient, both mentally and physically, to receive stem cell therapy; clear the nostrils for introduction of the stem cell specimen; introduce the stem cell specimen at a specific depth and angle in the inferior turbinate of the nostrils; and maintenance/monitor the patient after administration of the stem cell specimen, is still desired.

### SUMMARY

Illustrative embodiments of the disclosure are generally directed to a method for intranasal stem cell therapy. The method provides a non-invasive procedure to inject stem cells directly through the nostrils for stem cell deposition in nerve endings, which allows for the uptake of large number of stem cells directly into the central nervous system, and bypasses the blood-brain barrier.

The method involves a series of steps to prepare the patient, both mentally and physically, to receive stem cell therapy; conduct intravenous infusion of a substance that helps open up the blood brain barrier; clear the nostrils for introduction of the stem cell specimen; introduce the stem cell specimen at a specific depth and angle through the inferior turbinate of the nostrils; and maintenance/monitor the patient after administration of the stem cell specimen.

Specifically, the method involves: preparing the patient psychologically and physically prior to receiving the stem cell specimen with check-ups; intravenously introducing a solution of sodium chloride or Ringer's lactate containing mannitol, and later glutathione into the veins.

Further, the method also includes the steps of: clearing and illuminating the inferior turbinate of the nostrils; urging a pair of absorbent fiber soaked with lidocaine and epinephrine into the nostrils; inserting, through one of the nostrils, the 27G 1¼″ needle at a midpoint of the inferior turbinate at a 30° angle relative to the facial plane, and at an angle of about 5° to 10° relative to a lateral reference point, whereby the 27G ¼″ needle taps a middle turbinate bony structure after entering the inferior turbinate at a depth of about ½″; and performing post-procedure maintenance on the patient to determine efficacy and patient health.

The method may include an initial Step of obtaining at least one metric of a patient.

The method may further comprise a Step of inserting, through intravenous therapy, a solution of sodium chloride or Ringer's lactate from a 250 cubic centimeter intravenous bag, to the veins of the patient.

A Step includes drawing 25 cubic centimeters of the sodium chloride or Ringer's lactate from the intravenous bag into a 30 cubic centimeter syringe.

In some embodiments, a Step comprises injecting a dose of mannitol into the intravenous bag, the dose of mannitol comprising at least 0.25 g mannitol per kilogram of body weight, whereby the patient requires 1 milliliter of 25% mannitol per kilogram of body weight.

A Step includes infusing the entire contents of the intravenous bag into the veins of the patient.

In some embodiments, a Step may include drawing 1,000 milligrams of glutathione into the 30 cubic centimeter syringe containing the sodium chloride or Ringer's lactate.

A Step comprises injecting the entire contents of the 30 cubic centimeter syringe containing the glutathione and the sodium chloride or Ringer's lactate into the patient, whereby the glutathione helps to detoxify the body and serves as an antioxidant.

The method may further comprise a Step of soaking a plurality of absorbent fiber with 4 cubic centimeters of a solution consisting of lidocaine with epinephrine.

A Step includes urging the soaked absorbent fiber into a pair of nostrils of the patient.

A Step includes removing a stem cell specimen from dry ice, whereby the stem cell specimen thaws.

The method may further comprise a Step of drawing 2 cubic centimeters of the stem cell specimen into a 5 cubic centimeter syringe, the syringe being detachably attached to an 18G 1½″ needle.

A Step includes replacing the 18G 1½″ needle attached to the 5 cubic centimeter syringe with a 27G 1¼″ needle.

In some embodiments, a Step comprises removing the absorbent fiber from the nostrils.

A Step includes clearing a path through the inferior turbinate of the nostrils.

In some embodiments, a Step may include inserting, through one of the nostrils, the 27G 1¼″ needle at a midpoint of the inferior turbinate at a 30° angle relative to the facial plane, and at an angle of about 5° to 10° relative to a lateral reference point, whereby the 27G 1¼″ needle taps a middle turbinate bony structure after entering the inferior turbinate at a depth of about ½″.

Similarly, a Step comprises inserting, through the opposite nostril, the 27G 1¼″ needle at the midpoint of the inferior turbinate at a 30° angle relative to the facial plane, and at an angle of about 5° to 10° relative to a lateral reference point, whereby the 27G 1¼″ needle taps a middle turbinate bony structure after entering the inferior turbinate at a depth of about ½″.

The method may further comprise a Step of retreating the 27G 1¼″ needle about 1 to 2 millimeters from the middle turbinate bony structure.

A Step comprises injecting 1 cubic centimeter of the stem cell specimen into the inferior turbinate of each nostril.

A Step includes if resistance is felt while injecting the stem cell specimen, changing the orientation of the 27G 1¼″ needle.

A final Step includes massaging the areas next to the nostrils for about 1 minute to allow dispersion and absorption of the stem cell specimen.

In another aspect, the method further comprises a step of obtaining consent to proceed with the method from the patient.

In another aspect, the metric of a patient includes at least one of the following: body temperature, blood pressure, and pulse oximetry measurement.

In another aspect, the 1,000 milligrams of glutathione comprises 5 cc of 200 mg/ml glutathione.

In another aspect, the plurality of absorbent fiber comprises 2 cottonoid patties or cotton balls.

In another aspect, the method further comprises a step of soaking the absorbent fiber in a sterile container that contains the solution consisting of lidocaine with epinephrine.

In another aspect, the step of urging the soaked absorbent fiber into a pair of nostrils of the patient, further comprises the patient breathing steadily through the mouth for about 4 minutes while the soaked absorbent fiber are being urged.

In another aspect, the method further comprises a step of heating a vial containing the stem cell specimen to induce thawing by emerging the vial in a 37° water bath, or by rolling the vial against the palms of the hands.

In another aspect, the step of clearing a path through the interior of the nostrils is operable with a nasal speculum.

In another aspect, the method further comprises a step of illuminating the cleared path through the inferior turbinate of the nostrils.

In another aspect, the method further comprises a step of changing to a new 27G×1¼″ needle between injections of stem cell specimen.

In another aspect, the stem cell specimen comprises mesenchymal stem cells.

In another aspect, the method further comprises a step of allowing the patient to rest for about 5 to 10 minutes after injecting the stem cell specimen.

In another aspect, the method further comprises a step of measuring the blood pressure and oxygen saturation of the patient after injecting the stem cell specimen.

In another aspect, the method further comprises a step of performing a clinical evaluation of the patient prior to administering the method.

In another aspect, the method further comprises a step of performing multiple follow-up maintenance sessions for 3 to 12 months after administering the method.

One objective of the present invention is to use a patient's own stem cells to help repair damaged tissues and repair injuries; and deliver the stem cell through the nostrils, and specifically the inferior turbinate.

Another objective is to deliver therapeutic agents to the brain and spinal cord.

Another objective is to utilize intranasal stem cell procedure to enable stem cell deposition in areas that reach into nerve endings, allowing uptake of large number of stem cells directly into the central nervous system, and bypassing the blood-brain barrier.

Another objective is to prevent infections through use of a small sterile container; a Povidone-iodine swab sticks; two Cottonoid patties or cotton balls; and a 70% Isopropopyl alcohol bottle.

Another objective is to ensure the mental well-being of the patient prior to the stem cell therapy.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIGS. 1A and 1B illustrates a flowchart diagram of an exemplary method for intranasal stem cell therapy, in accordance with an embodiment of the present invention;

FIG. 2 illustrates a frontal view of a human head, showing the nostrils and inferior turbinate, in accordance with an embodiment of the present invention;

FIG. 3 illustrates a perspective view of a nostril being pried pen for introduction of a soaked resilient fiber, in accordance with an embodiment of the present invention;

FIG. 4 illustrates a sectioned side view of a human head, showing a needle positioned at a 30° angle relative to the facial plane, and at an angle of about 5° to 10° relative to a lateral reference point, in accordance with an embodiment of the present invention; and

FIG. 5 illustrates a sectioned side view of a nostril, showing the needle injecting a stem cell specimen into the inferior turbinate, in accordance with an embodiment of the present invention.

Like reference numerals refer to like parts throughout the various views of the drawings.

### DETAILED DESCRIPTION OF THE INVENTION

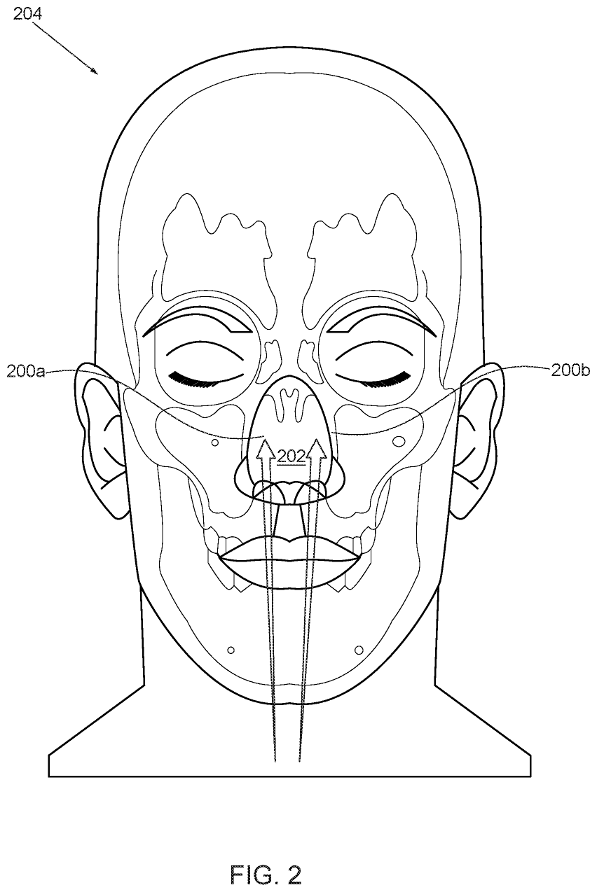

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 2. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

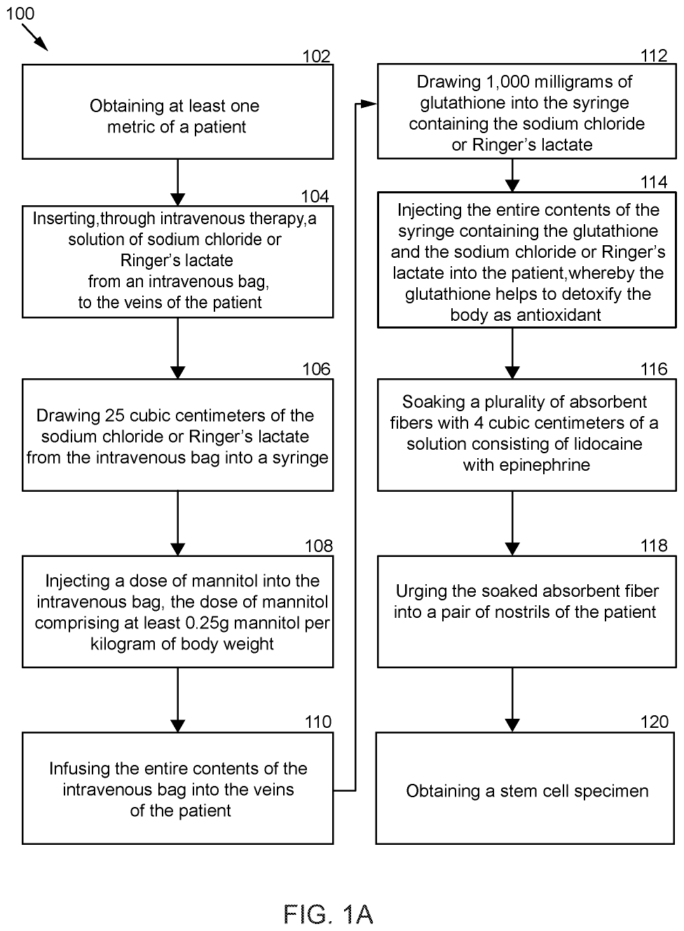

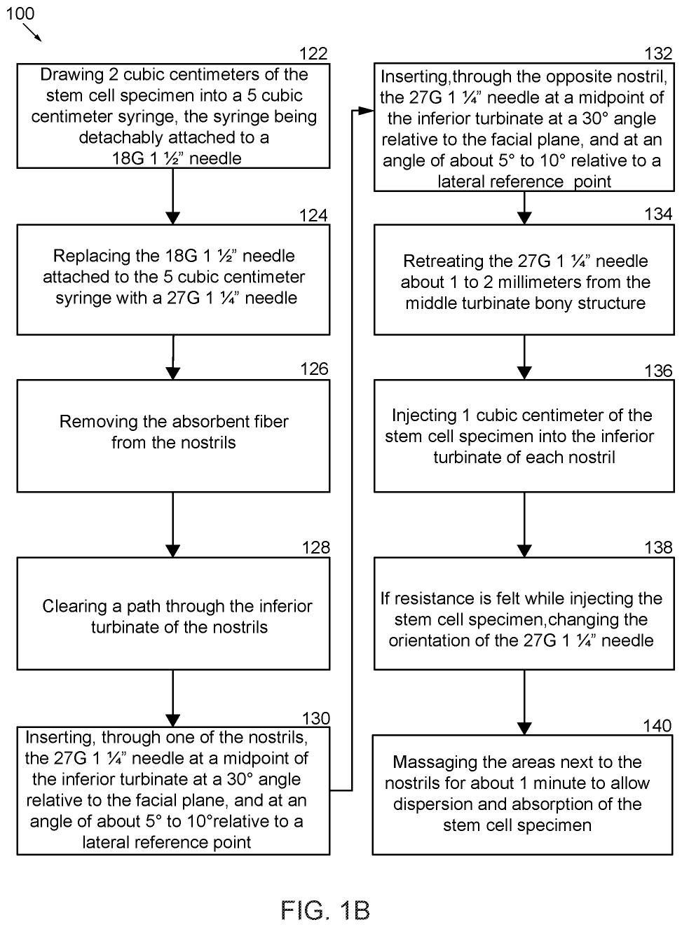

A method 100 for intranasal stem cell therapy is referenced in FIGS. 1A-5. The method 100 provides a non-invasive procedure to inject a stem cell specimen 500, such as mesenchymal stem cells, directly through the nostrils 200. The unique capacity to deliver stem cells directly into the nostrils 200 at a specific angle and depth in the inferior turbinate 202 provides numerous advantages discussed below.

In some embodiments, the method 100 provides stem cell deposition in nerve endings, which allows for the uptake of large number of stem cells directly into the central nervous system, and bypasses the blood-brain barrier. This is possible because of the methodical intranasal delivery means that prepare the patient 204 for intranasal administration of the stem cell specimen 500. Further, the method 100 has been experimentally shown to be efficacious for treating Parkinson's disease and neonatal ischemic brain damage (See Appendix A and B).

The method 100 involves a series of steps to prepare the patient 204, both mentally and physically, to receive stem cell therapy. The method 100 also involves intravenous introduction of antioxidants into the veins prior to introducing the stem cell specimen 500. The method 100 also requires the nostrils 200 to be cleared and illuminated prior to injecting the stem cell specimen 500 therein. Through a specifically sized needle, a syringe injects the stem cell specimen 500 at a specific depth and angle in the inferior turbinate 202 of the nostrils 200. After administering the stem cell specimen 500, the patient 204 is monitored and check on periodically for 3 to 12 months.

Specifically, the method 100 involves: preparing the patient 204 psychologically and physically prior to receiving the stem cell specimen 500 with mental wellness check-ups and body metric measurements; and intravenously introducing a solution of sodium chloride or Ringer's lactate which contain mannitol, and then glutathione into the veins. Further, the method 100 includes: clearing and illuminating the inferior turbinate 202 of the nostrils 200 with a nasal speculum; urging a pair of absorbent fiber soaked with lidocaine and epinephrine into the nostrils 200; inserting, through one of the nostrils 200, a 27G 1¼″ needle at a midpoint of the inferior turbinate 202 at a 30° angle relative to the facial plane, and at an angle of about 5° to 10° relative to a lateral reference point. In this position, the 27G 1¼″ needle taps a middle turbinate bony structure after entering the inferior turbinate 202 at a depth of about ½″. The method also includes performing post-procedure maintenance on the patient to determine efficacy and patient health. In this manner, a patient's own stem cells can be used to repair damaged tissues and heal injuries through efficient delivery of the stem cell specimen 500 through the inferior turbinate 202 of the nostrils 200.

The method 100, as described below, requires various medical instruments and solutions to properly administer the stem cell specimen 500 through intranasal means. The medical instruments and solutions may include, without limitation, a ChareCore™ cell specimen 2cc, which is in essence, the stem cell specimen 500. It is known in the art that CharaCore™ contains 8-10 million cells/cc. These are “native stem cells.” Research shows that native mesenchymal stem cells (MSC's) are at least 10 times more potent than expanded MSC's. Further, Characore™ exhibits a high cell count, with valuable components from umbilical cord blood, cord tissue and amniotic membrane. Delicately processed, thus maximally preserving therapeutic elements. Characore™ contains a high amount of MSC's and various growth factors. Characore™ is offered in 1cc and 2 cc vials for all forms of injections; through the present invention utilizes the 2cc vial −1cc for each nostril.

The method 100 also requires two 25% mannitol 50 ml bottles for injection into the patient prior to administrating the stem cell specimen 500. However the mannitol bottles should be avoided if the patient has kidney failure or severe pulmonary congestion/edema. Additional solutions include 0.9% Normal Saline (NS) or Lactated Ringer (LR) 250 ml. Another solution required for the method 100 is Lidocaine 2% with epinephrine 1:100,000. Yet another solution is Glutathione 200 mg/ml. However, different volumes and concentrations of solutions may also be used in other embodiments.

Furthermore, to administer the stem cell specimen 500 intranasally, various syringes are used, including: a 5 cc syringe×1; a 30 cc syringe×2; 27G×1¼″ needles×2; a 5½″ needle holder×1; a 18G×1½″ drawing needle×1; and a Vienna nasal speculum×1. Though different dimensions of needles and syringes may be used to accommodate different sized nostrils 200. A small medical flashlight may also be used to enhance viewing inside the nasal cavity for introduction of the syringe. One objective of the method 100 is to prevent infections. Thus, the method 100 may also require: a small sterile container for soaking the absorbent fiber; a Povidone-iodine swabsticks; two Cottonoid patties or cotton balls; and a 70% Isopropopyl alcohol bottle.

FIGS. 1A and 1B illustrate a flowchart diagram of an exemplary method 100 for intranasal stem cell therapy. The method 100 may include an initial Step 102 of obtaining at least one metric of a patient 204. The metric of a patient 204 may include, without limitation, body temperature, blood pressure, and pulse oximetry measurement. Though in other embodiments, different metrics may be measured. In some embodiments, the method 100 also includes a step of obtaining consent to proceed with the method 100 from the patient. Various legal forms known in the art may be used for documenting the consent.

The method 100 requires a clinical evaluation of the patient to determine appropriateness for stem cell therapy. The clinic collects a detailed medical history and provides a physical exam of the patient. The patient next undergoes a basic laboratory prescreen, checking the CBC, Chem, LFT's, Lipids, and CRP. As FIG. 2 shows, the nostrils 200 of the patient 204 are the central body part through which the stem cell specimen 500 is administered. Thus, the nostrils 200 may also be examined to determine suitable syringe sized and positioning therein.

In alternative embodiments of the method 100, the patient 204 may also receive a Mini Mental State Exam if the patient has a history of cognitive impairment. This examination is performed prior to each treatment session to track progress in cognitive functioning. In another alternative embodiment, the method 100 may include a step of performing a clinical evaluation of the patient prior to administering the method 100.

Continuing, the method 100 further includes a Step 104 of inserting, through intravenous therapy, a solution of sodium chloride or Ringer's lactate from a 250 cubic centimeter intravenous bag, to the veins of the patient. The intravenous introduction of these solutions helps prepare the body for the stem cell specimen 500.

A Step 106 includes drawing 25 cubic centimeters of the sodium chloride or Ringer's lactate from the intravenous bag into a 30 cubic centimeter syringe. In some embodiments, a Step 108 comprises injecting a dose of mannitol into the intravenous bag, the dose of mannitol comprising at least 0.25 g mannitol per kilogram of body weight, whereby the patient requires 1 milliliter of 25% mannitol per kilogram of body weight. The mannitol is useful for preventing infections, complications, and for receiving the needle and stem cell specimen.

However, it is significant to note that the mannitol solution may be omitted from the method 100 if the patient has kidney failure or severe pulmonary congestion/edema. In some embodiments, a Step 110 includes infusing the entire contents of the intravenous bag into the veins of the patient. This is performed through standard drip-intravenous means known in the art.

A Step 112 may include drawing 1,000 milligrams of glutathione into the 30 cubic centimeter syringe containing the sodium chloride or Ringer's lactate. In one non-limiting embodiment, the 1,000 milligrams of glutathione comprises 5 cc of 200 mg/ml glutathione. A Step 114 comprises injecting the entire contents of the 30 cubic centimeter syringe containing the glutathione and the sodium chloride or Ringer's lactate into the patient, whereby the glutathione helps to detoxify the body and serves as an antioxidant. In any case, a medical professional can determine the concentration and need of the sodium chloride or Ringer's lactate.

The method 100 may further comprise a Step 116 of soaking a plurality of absorbent fibers 302 with 4 cubic centimeters of a solution consisting of lidocaine with epinephrine. This solution helps anesthetize the area. The solution can be placed in a sterile container, from which the absorbent fiber are soaked. In some embodiments, the plurality of absorbent fibers may include, without limitation, 2 cottonoid patties or cotton balls. However, any medical-grade absorbent material that can be pushed into the nostrils 200 may also be used.

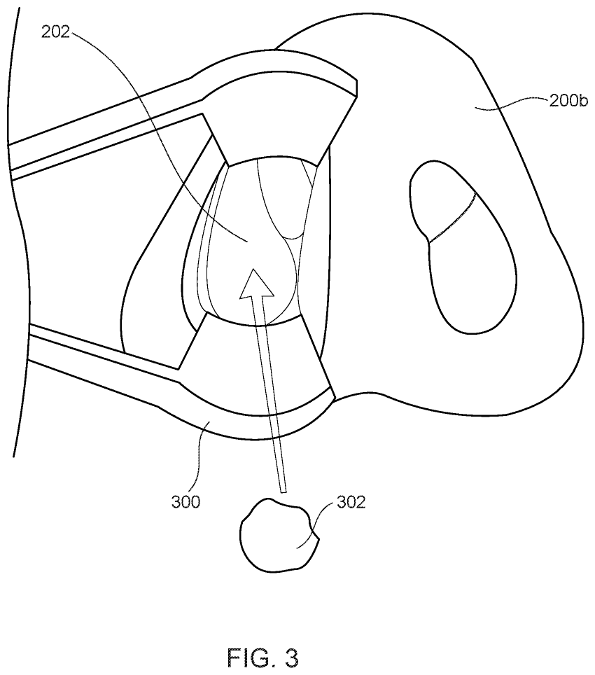

As FIG. 3 shows, a Step 118 includes urging the soaked absorbent fiber 302 into a pair of nostrils 200 of the patient. This may be performed with a needle holder. While pushing the absorbent fiber through, the patient breathes steadily through the mouth for about 4 minutes simultaneously with the soaked absorbent fiber being urged through the nostrils 200.

A Step 120 includes removing a stem cell specimen 500 from dry ice, whereby the stem cell specimen 500 thaws. The stem cell specimen 500 may include 2cc's of CharaCore, which has a high percentage of mesenchymal stem cells. Though in other embodiments, different stem cells form the patient or relatives thereof may also be used.

It is known in the art that mesenchymal stem cells (MSCs) represents a class of cells that has the in vitro capacity to be induced to form mesodermal tissues, such as bone, cartilage, fat, etc. MSC are multipotent stromal cells that can differentiate into a variety of cell types, including osteoblasts (bone cells), chondrocytes (cartilage cells), myocytes (muscle cells) and adipocytes (fat cells which give rise to marrow adipose tissue). This Step 120 may also include heating a vial containing the stem cell specimen 500 to induce thawing. This can be performed by either emerging the vial in a 37° water bath, or by rolling the vial against the palms of the hands.

The method 100 may further comprise a Step 122 of drawing 2 cubic centimeters of the stem cell specimen 500 into a 5 cubic centimeter syringe, the syringe being detachably attached to an 18G 1½″ needle. A Step 124 includes replacing the 18G 1½″ needle attached to the 5 cubic centimeter syringe with a 27G 1¼″ needle. However in other embodiments, different combinations and sizes of needles may be used. Further, in one embodiment, it is not necessary to change the size of the needle.

In some embodiments, a Step 126 comprises removing the absorbent fiber from the nostrils 200. This indicates that the stem cell specimen 500 is soon to be administered through the described intranasal means. FIG. 3 illustrates a Step 128 of clearing a path through the inferior turbinate 202 of the nostrils 200 with a nasal speculum 300 or other medical instrument known in the art and used for this purpose. In another embodiment, the method 100 comprises a step of illuminating the cleared path through the inferior turbinate 202 of the nostrils 200. This illumination of the nostrils 200 may be performed by a hand-held medical flashlight.

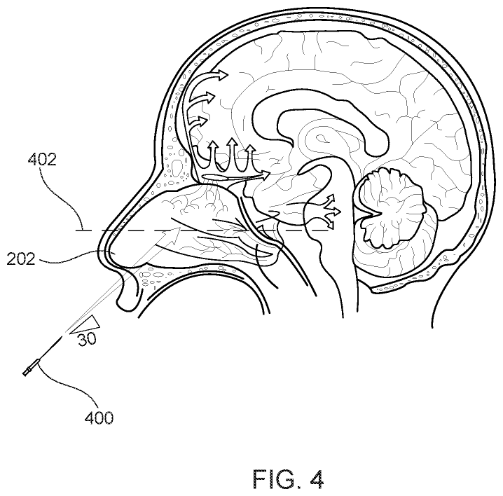

In some embodiments, a Step 130 may include inserting, through one of the nostrils 200, the 27G 1¼″ needle 400 at a midpoint of the inferior turbinate 202 at a 30° angle relative to the facial plane, and at an angle of about 5° to 10° relative to a lateral reference point, whereby the 27G 1¼″ needle taps a middle turbinate bony structure after entering the inferior turbinate 202 at a depth of about ½″. This specific angle and depth is shown in FIG. 4, and is vital for proper stem cell deposition. However in some embodiments, the angle and depth can be adjusted to accommodate uniquely sized and shaped nostrils 200.

A Step 132 comprises inserting, through the opposite nostril, the 27G 1¼″ needle 400 at the midpoint of the inferior turbinate 202 at a 30° angle relative to the facial plane, and at an angle of about 5° to 10° relative to a lateral reference point 402 (see FIG. 4). The 27G 1¼″ needle 400 is positioned to tap a middle turbinate bony structure after entering the inferior turbinate 202 at a depth of about ½″. This provides a tactile indication of the appropriate position for stem cell deposition. Thus, both nostrils 200 are injected with the stem cell specimen 500. The method 100 may further comprise a Step 134 of retreating the 27G 1¼″ needle 400 about 1 to 2 millimeters from the middle turbinate bony structure. The medical professional can feel the appropriate depth through tactile sensation.

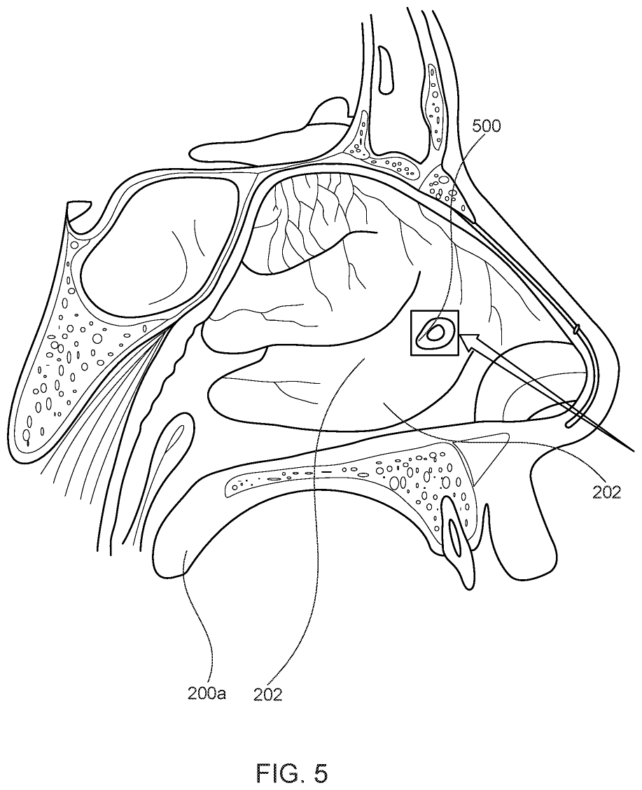

As shown in FIG. 5, a Step 136 comprises injecting 1 cubic centimeter of the stem cell specimen 500 into the inferior turbinate 202 of each nostril. The injection occurs after the positioning of the needle and the retreat to the specified depth and angle. A Step 138 includes, if resistance is felt while injecting the stem cell specimen 500, changing the orientation of the 27G 1¼″ needle. This change is generally less than an inch, or a few degrees. In some embodiments, the method 100 further comprises a step of changing to a new 27G×1¼″ needle between injections of stem cell specimen 500. This helps prevent cross-contamination.

As discussed above, the method 100 is advantageous in that the stem cell specimen 500—being delivered through intranasal means—does not have to cross the blood-brain barrier to be delivered to the central nervous system. This delivery is generally finished quickly—within minutes. The intranasal means also directly delivers drugs that do blood-brain barrier the blood-brain barrier to the brain, eliminating the need for systemic administration and its potential side effects. This is possible because of the unique connections that the olfactory and trigeminal nerves provide between the brain and external environment.

An Initial Phase of the method 100 generally requires 1-3 sessions, about 4 weeks apart. For each Initial Phase session, 2 cc's of CharaCore is required. Note: Intranasal stem cell procedure allows stem cell deposition in area reach into nerve endings, allowing uptake of large number of stem cells directly into the central nervous system, bypassing the blood-brain barrier (see Olfactory and Trigeminal pathways in FIG. 4).

A final Step 140 includes massaging the areas next to the nostrils 200 for about 1 minute to allow dispersion and absorption of the stem cell specimen 500. The massaging motion helps stimulate the stem cell specimen 500, and circulate blood flow through the nostrils 200. In some embodiments, the method 100 may include an alternative step of allowing the patient to rest for about 5 to 10 minutes after injecting the stem cell specimen 500.

After the Initial Phase, the method 100 utilizes a Maintenance Phase. Here, the date of follow-up session depends on how well the patient is doing, and could range between 3 to 12 months. Each session is at 2 cc dosage. In the Maintenance Phase, the method 100 requires measuring the blood pressure and oxygen saturation of the patient after injecting the stem cell specimen 500. Further, the method 100 may also include a step of performing multiple follow-up maintenance sessions for 3 to 12 months after administering the stem cell specimen 500.

It is recognized in the art that stem cell therapy is a complex and potentially dangerous procedure. Thus, the method may further include alternative Steps of providing emergency supplies in the event of untoward reactions. One such step is providing an Ammonium salt ampule for sniffing in case of vasovagal reaction. It may also be beneficial to provide Epinephrine 1:1000 (1 mg/ml), inject 0.5 mg IV or IM in case of anaphylactic reaction. The Epinephrine administration may repeat in 5 minutes if needed. Yet another emergency supply may include Benadryl 50 mg IV, which can be pushed over 2 minutes for anaphylactic reactions (may give after epinephrine dose). Finally, an Albuterol inhaler is provided in case the patient demonstrates shortness of breath during or after the method is applied.

In one experimental application of the method 100, detailed in Appendix A, the Therapeutic Efficacy of Intranasally Delivered Mesenchymal Stem Cells in a Rat Model of Parkinson Disease is taught. In this Parkinson disease experiment, intranasal application of MSCs resulted in the appearance of cells in the olfactory bulb, cortex, hippocampus, striatum, cerebellum, brainstem, and spinal cord. Out of 1 million MSCs injected, 24% survived for at least 4.5 months in the brains of 6-OHDA rats. Further, 3% of applied MSC's were proliferative at 4.5 months. Thus, the intranasal application of MSCs prevented any decrease in the dopamine level in the lesioned hemisphere, whereas the lesioned side of the control animals revealed significantly lower levels of dopamine 4.5 months later. Behavioral analyses revealed significant and substantial improvement of motor function.

Turning now to Appendix B, in another experiment performed with the method 100, it was discovered that nasal administration of stem cells (intranasal method) provided a novel route for treatment of Neonatal Ischemic Brain Damage. In this experiment, 9-day-old mice underwent cerebral hypoxia-ischemia (HI), and MSCs transplanted intranasally 10 days after HI. Furthermore, at 28 days after HI, MSCs were still present in the affected hemisphere but had not differentiated into cerebral cell types. In this experiment, the intranasal MSC treatment significantly improved sensorimotor function at 21 and 28 d after HI. MSC cultured in vitro with brain extracts obtained 10 days after HI, responded to the ischemic brain by up-regulation of several growth factors. It was discovered that the MSCs migrate to the lesion site not only from the ipsilateral but also the contralateral hemisphere.

These and other advantages of the invention will be further understood and appreciated by those skilled in the art by reference to the following written specification, claims and appended drawings.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. A method for intranasal stem cell therapy, the method comprising:
obtaining at least one metric of a patient;
inserting, through intravenous therapy, a solution of sodium chloride or Ringer's lactate from an intravenous bag, to the veins of the patient;
drawing 25 cubic centimeters of the sodium chloride or Ringer's lactate from the intravenous bag into a syringe;
injecting a dose of mannitol into the intravenous bag, the dose of mannitol comprising at least 0.25 g mannitol per kilogram of body weight;
infusing the entire contents of the intravenous bag into the veins of the patient;
drawing 1,000 milligrams of glutathione into the syringe containing the sodium chloride or Ringer's lactate;
injecting the entire contents of the syringe containing the glutathione and the sodium chloride or Ringer's lactate into the patient, whereby the glutathione helps to detoxify the body and serves as an antioxidant;
soaking a plurality of absorbent fibers with 4 cubic centimeters of a solution consisting of lidocaine with epinephrine;
urging the soaked absorbent fiber into a pair of nostrils of the patient;
obtaining a stem cell specimen;
drawing 2 cubic centimeters of the stem cell specimen into a 5 cubic centimeter syringe, the syringe being detachably attached to a 18G 1½″ needle;
replacing the 18G 1½″ needle attached to the 5 cubic centimeter syringe with a 27G 1¼″ needle;
removing the absorbent fiber from the nostrils;
clearing a path through the inferior turbinate of the nostrils;
inserting, through one of the nostrils, the 27G 1¼″ needle at a midpoint of the inferior turbinate at a 30° angle relative to the facial plane, and at an angle of about 5° to 10° relative to a lateral reference point, whereby the 27G 1¼″ needle taps a middle turbinate bony structure after entering the inferior turbinate at a depth of about ½″;
inserting, through the opposite nostril, the 27G 1¼″ needle at the midpoint of the inferior turbinate at a 30° angle relative to the facial plane, and at an angle of about 5° to 10° relative to a lateral reference point, whereby the 27G 1¼″ needle taps a middle turbinate bony structure after entering the inferior turbinate at a depth of about ½″;
retreating the 27G 1¼″ needle about 1 to 2 millimeters from the middle turbinate bony structure;
injecting 1 cubic centimeter of the stem cell specimen into the inferior turbinate of each nostril;
if resistance is felt while injecting the stem cell specimen, changing the orientation of the 27G 1¼″ needle; and
massaging the areas next to the nostrils for about 1 minute to allow dispersion and absorption of the stem cell specimen.

2. The method of claim 1, further comprising a step of obtaining consent from the patient to proceed with the method.

3. The method of claim 1, wherein the metric of a patient includes at least one of the following: body temperature, blood pressure, and pulse oximetry measurement.

4. The method of claim 1, wherein the 1,000 milligrams of glutathione comprises 5 cubic centimeters of 200 mg/ml glutathione.

5. The method of claim 1, wherein the plurality of absorbent fiber comprises 2 cottonoid patties or cotton balls.

6. The method of claim 1, further comprising a step of soaking the absorbent fiber in a sterile container that contains the solution consisting of lidocaine with epinephrine.

7. The method of claim 1, wherein the step of urging the soaked absorbent fiber into a pair of nostrils of the patient, further comprises the patient breathing steadily through the mouth for about 4 minutes while the soaked absorbent fiber are being urged.

8. The method of claim 1, further comprising a step of heating a vial containing the stem cell specimen to induce thawing by emerging the vial in a 37° water bath, or by rolling the vial against the palms of the hands.

9. The method of claim 1, wherein the step of clearing a path through the interior of the nostrils is operable with a nasal speculum.

10. The method of claim 1, further comprising a step of illuminating the cleared path through the inferior turbinate of the nostrils.

11. The method of claim 1, wherein the stem cell specimen comprises a mesenchymal stem cell specimen.

12. The method of claim 1, further comprising a step of changing to a new 27G×1¼″ needle between injections of stem cell specimen.

13. The method of claim 1, further comprising a step of allowing the patient to rest for about 5 to 10 minutes after injecting the stem cell specimen.

14. The method of claim 1, further comprising a step of measuring the blood pressure and oxygen saturation of the patient after injecting the stem cell specimen.

15. The method of claim 1, further comprising a step of performing a clinical evaluation of the patient prior to injecting the stem cell specimen.

16. The method of claim 1, further comprising a step of performing multiple follow-up maintenance sessions for 3 to 12 months after injecting the stem cell specimen.

17. A method for intranasal stem cell therapy, the method comprising:
performing a clinical evaluation of the patient prior to administering the method;
obtaining at least one metric of a patient;
inserting, through intravenous therapy, a solution of sodium chloride or Ringer's lactate from a 250 cubic centimeter intravenous bag, to the veins of the patient;
drawing 25 cubic centimeters of the sodium chloride or Ringer's lactate from the intravenous bag into a 30 cubic centimeter syringe;
injecting a dose of mannitol into the intravenous bag, the dose of mannitol comprising at least 0.25 g mannitol per kilogram of body weight, whereby the patient requires 1 milliliter of 25% mannitol per kilogram of body weight;
infusing the entire contents of the intravenous bag into the veins of the patient;
drawing 1,000 milligrams of glutathione into the 30 cubic centimeter syringe containing the sodium chloride or Ringer's lactate;
injecting the entire contents of the 30 cubic centimeter syringe containing the glutathione and the sodium chloride or Ringer's lactate into the patient, whereby the glutathione helps to detoxify the body and serves as an antioxidant;
soaking a plurality of absorbent fiber with 4 cubic centimeters of a solution consisting of lidocaine with epinephrine;
urging the soaked absorbent fiber into a pair of nostrils of the patient;
removing a stem cell specimen from dry ice, whereby the stem cell specimen thaws;
drawing 2 cubic centimeters of the stem cell specimen into a 5 cubic centimeter syringe, the syringe being detachably attached to a 18 G 1½″ needle;
replacing the 18G 1½″ needle attached to the 5 cubic centimeter syringe with a 27G 1¼″ needle;
removing the absorbent fiber from the nostrils;
clearing a path through the inferior turbinate of the nostrils;
illuminating the cleared path through the inferior turbinate of the nostrils;
inserting, through one of the nostrils, the 27G 1¼″ needle at a midpoint of the inferior turbinate at a 30° angle relative to the facial plane, and at an angle of about 5° to 10° relative to a lateral reference point, whereby the 27G 1¼″ needle taps a middle turbinate bony structure after entering the inferior turbinate at a depth of about ½″;
inserting, through the opposite nostril, the 27G 1¼″ needle at the midpoint of the inferior turbinate at a 30° angle relative to the facial plane, and at an angle of about 5° to 10° relative to a lateral reference point, whereby the 27G 1¼″ needle taps a middle turbinate bony structure after entering the inferior turbinate at a depth of about ½″;
retreating the 27G 1¼″ needle about 1 to 2 millimeters from the middle turbinate bony structure;
injecting 1 cubic centimeter of the stem cell specimen into the inferior turbinate of each nostril;
if resistance is felt while injecting the stem cell specimen, changing the orientation of the 27G 1¼″ needle;
massaging the areas next to the nostrils for about 1 minute to allow dispersion and absorption of the stem cell specimen;
allowing the patient to rest for about 5 to 10 minutes after injecting the stem cell specimen; and
performing multiple follow-up maintenance sessions for 3 to 12 months after injecting the stem cell specimen.

18. The method of claim 17, wherein the stem cell specimen comprises a mesenchymal stem cell specimen.

19. The method of claim 17, wherein the step of urging the soaked absorbent fiber into a pair of nostrils of the patient, further comprises the patient breathing steadily through the mouth for about 4 minutes while the soaked absorbent fiber are being urged.

20. A method for intranasal stem cell therapy, the method comprising:
performing a clinical evaluation of the patient prior to administering the method;
obtaining at least one metric of a patient, the metric of a patient including at least one of the following: body temperature, blood pressure, and pulse oximetry measurement;
inserting, through intravenous therapy, a solution of sodium chloride or Ringer's lactate from a 250 cubic centimeter intravenous bag, to the veins of the patient;
drawing 25 cubic centimeters of the sodium chloride or Ringer's lactate from the intravenous bag into a 30 cubic centimeter syringe;
injecting a dose of mannitol into the intravenous bag, the dose of mannitol comprising at least 0.25 g mannitol per kilogram of body weight, whereby the patient requires 1 milliliter of 25% mannitol per kilogram of body weight;
infusing the entire contents of the intravenous bag into the veins of the patient;
drawing 1,000 milligrams of glutathione into the 30 cubic centimeter syringe containing the sodium chloride or Ringer's lactate;
injecting the entire contents of the 30 cubic centimeter syringe containing the glutathione and the sodium chloride or Ringer's lactate into the patient, whereby the glutathione helps to detoxify the body and serves as an antioxidant;
soaking a plurality of absorbent fiber with 4 cubic centimeters of a solution consisting of lidocaine with epinephrine;
urging the soaked absorbent fiber into a pair of nostrils of the patient, whereby the patient breathes steadily through the mouth for about 4 minutes while the soaked absorbent fiber are being urged;
removing a mesenchymal stem cell specimen from dry ice;
heating a vial containing the stem cell specimen to induce thawing by emerging the vial in a 37° water bath, or by rolling the vial against the palms of the hands;
drawing 2 cubic centimeters of the mesenchymal stem cell specimen into a 5 cubic centimeter syringe, the syringe being detachably attached to a 18 G 1½″ needle;
replacing the 18G 1½″ needle attached to the 5 cubic centimeter syringe with a 27G 1¼″ needle;
removing the absorbent fiber from the nostrils;
clearing a path through the inferior turbinate of the nostrils with a nasal speculum;
illuminating the cleared path through the inferior turbinate of the nostrils;
inserting, through one of the nostrils, the 27G 1¼″ needle at a midpoint of the inferior turbinate at a 30° angle relative to the facial plane, and at an angle of about 5° to 10° relative to a lateral reference point, whereby the 27G 1¼″ needle taps a middle turbinate bony structure after entering the inferior turbinate at a depth of about ½″;
inserting, through the opposite nostril, the 27G 1¼″ needle at the midpoint of the inferior turbinate at the 30° angle relative to the facial plane, and at the angle of about 5° to 10° relative to the lateral reference point, whereby the 27G 1¼″ needle taps a middle turbinate bony structure after entering the inferior turbinate at a depth of about ½″;
retreating the 27G 1¼″ needle about 1 to 2 millimeters from the middle turbinate bony structure;
injecting 1 cubic centimeter of the mesenchymal stem cell specimen into the inferior turbinate of each nostril;
if resistance is felt while injecting the mesenchymal stem cell specimen, changing the orientation of the 27G 1¼″ needle;
massaging the areas next to the nostrils for about 1 minute to allow dispersion and absorption of the mesenchymal stem cell specimen;
allowing the patient to rest for about 5 to 10 minutes after injecting the mesenchymal stem cell specimen; and
performing multiple follow-up maintenance sessions for 3 to 12 months after injecting the mesenchymal stem cell specimen.
